(19) United States
(12) Patent Application Publication
(10) Pub. No.: US 2024/0335837 A1
(43) Pub. Date: Oct. 10, 2024

(54) DEVICES AND METHODS FOR SAMPLE DISSOCIATION AND MANIPULATION IN A MICROFLUIDIC SYSTEM

(71) Applicant: TDK U.S.A. Corporation, Uniondale, NY (US)

(72) Inventors: Manish Giri, San Jose, CA (US); Rakesh Sethi, San Jose, CA (US); Vadim Piskun, San Jose, CA (US)

(73) Assignee: TDK U.S.A. Corporation, Uniondale, NY (US)

(21) Appl. No.: 18/605,476

(22) Filed: Mar. 14, 2024

Related U.S. Application Data
(60) Provisional application No. 63/467,903, filed on May 19, 2023.
(60) Provisional application No. 63/457,507, filed on Apr. 6, 2023.

(51) Int. Cl. B01L 3/00

(52) U.S. Cl. CPC B01L 3/502761; B01L 2200/0647; B01L 2300/0663; B01L 2300/0864; B01L 2400/0436; B01L 2400/0496

(57) ABSTRACT

A microfluidic device and a method for dissociating and manipulating cells or particles in a microfluidic channel are disclosed. The microfluidic device includes an inlet, an outlet, and a microfluidic channel arranged on a substrate between the inlet and the outlet. The microfluidic device includes a first set of piezoelectric actuators arranged adjacent to the inlet channel and configured to dissociate particles of a fluidic sample in the microfluidic channel. The microfluidic device includes a second set of piezoelectric actuators arranged between the inlet channel and the outlet channel and configured to manipulate the particles of the fluidic sample as the particles move through the microfluidic channel. The microfluidic device includes a third set of piezoelectric actuators arranged above the outlet channel, adjacent to the outlet, and configured to eject a portion of the fluidic sample out of the microfluidic channel via the outlet.

### PRIORITY AND RELATED APPLICATIONS

This application claims priority to U.S. Provisional Patent Ser. No. 63/467,903, filed May 19, 2023, entitled “Microfluidic chip for 3D scanning of single cells using piezoelectric actuators in a sensing zone,” and U.S. Provisional Patent App. Ser. No. 63/457,507, filed Apr. 6, 2023, entitled “Integrated microfluidics chip with Plurality of individually controllable thin film piezoelectric vibrating devices for sample preparation, sorting, levitation, rotation, regulating flow and droplet formation,” each of which is incorporated by reference herein in its entirety.

### TECHNICAL FIELD

This application relates generally to flow control of cells or particles, and more particularly to dissociation and manipulation of particles in a microfluidic flow channel.

### BACKGROUND

Conventional sample preparation for analysis of single-cell samples requires chemical (enzymatic) processes, mechanical processes, and careful handling. Specifically, for microfluidics-based single cell analysis techniques, it is important that the input sample have no clumping since that would render a microfluidic chip useless. Therefore, it is important to have a mechanism that can dissociate cell samples before the sample is inserted into the microfluidic chip. Centrifugation is the most common method to mechanically disrupt clumps, and, on a microscopic scale, Surface Acoustic Waves (SAW), and Bulk Acoustic Waves (BAW) have also been demonstrated to disrupt clumps. These methods have significant limitations as centrifugation is a macro-scale process and is an additional step which requires dilution followed by verification and can sometime harm fragile cells. SAW and BAW require high power, are very slow, have a limited regime of operation (they cannot effectively cause flow and are easily overwhelmed by lateral flows) and have been shown to not be easily integrated into microfluidic devices for manufacturing.

### SUMMARY

As described above, dissociating cell samples is a challenging problem in microfluidic devices. In some systems, centrifugation, SAW, and BAW have been demonstrated to disrupt clumps. However, these methods pose challenges to microfluidic chip design, in particular, they are not easily integrated into microfluidic devices for manufacturing. Devices and methods for dissociating and manipulating cell samples in microfluidic devices and systems are described herein. Such devices and methods may address challenges associated with conventional devices and methods for dissociating and manipulating cell samples in microfluidic devices or systems.

For example, a particle sensing system (e.g., for cells, bacteria, and/or viruses) with integrated sets of piezoelectric actuators (e.g., that form a laminar flow in the microfluidic device) is described. The sets of piezoelectric actuators dissociate and manipulate particles of a fluidic sample. An additional set of piezoelectric actuators can eject at least a portion of the particles and/or fluidic sample from the microfluidic device.

In accordance with some embodiments, a microfluidic device includes: (i) an inlet channel, (ii) an outlet channel, (iii) a microfluidic channel, (iv) a first set of piezoelectric actuators, (v) a second set of piezoelectric actuators, (vi) a third set of piezoelectric actuators. The microfluidic channel is arranged a substrate between the inlet channel and the outlet channel such that an outlet of the microfluidic channel is positioned above at least a portion of the outlet channel. The first set of piezoelectric actuators is arranged adjacent to the inlet channel and configured to dissociate particles of a fluidic sample in the microfluidic channel. The second set of piezoelectric actuators is arranged between the inlet channel and the outlet channel. The second set of piezoelectric actuators is configured to manipulate the particles of the fluidic sample as the particles move through the microfluidic channel. The third set of piezoelectric actuators is arranged above the outlet channel and is adjacent to the outlet. The third set of piezoelectric actuators is configured to eject a portion of the fluidic sample out of the microfluidic channel via the outlet.

In accordance with some embodiments, a method includes (i) providing a fluidic sample comprising plurality of particles through an inlet to a microfluidic channel of a microfluidic device, the microfluidic channel having an outlet, (ii) selectively dissociating two or more particles of the fluidic sample using a first set of piezoelectric actuators positioned adjacent to the inlet, (iii) selectively manipulating, using a second set of piezoelectric actuators, one or more particles of the fluidic sample flowing through the microfluidic channel, and (iv) selectively ejecting, with a third set of piezoelectric actuators located adjacent to the outlet, a portion of the fluidic sample from the microfluidic channel.

Thus, the disclosed devices and methods relate to techniques for dissociating and manipulating cell samples which are implemented within or as part of a microfluidic device using sets piezoelectric actuators. Such techniques provide reliable preparation, localization, and analysis of single-cell samples. The disclosed devices and methods may replace, or complement, conventional devices and methods.

The features and advantages described in the specification are not necessarily all-inclusive and, in particular, some additional features and advantages will be apparent to one of ordinary skill in the art in view of the drawings, specification, and claims provided in this disclosure. Moreover, it should be noted that the language used in the specification has been principally selected for readability and instructional purposes and has not necessarily been selected to delineate or circumscribe the subject matter described herein.

### BRIEF DESCRIPTION OF THE DRAWINGS

So that the present disclosure can be understood in greater detail, a more particular description can be had by reference to the features of various embodiments, some of which are illustrated in the appended drawings. The appended drawings, however, merely illustrate pertinent features of the present disclosure and are therefore not necessarily to be considered limiting, for the description can admit to other effective features as the person of skill in this art will appreciate upon reading this disclosure.

FIG. 1A shows a plan view of an example microfluidic device, in accordance with some embodiments.

FIG. 1B shows a cross-section view of the microfluidic device of FIG. 1A, in accordance with some embodiments.

FIG. 1C shows a plan view and a corresponding cross-sectional view of an input region of the microfluidic device of FIG. 1A, in accordance with some embodiments.

FIG. 1D shows a plan view and a corresponding cross-sectional view of a regulation region of the microfluidic device of FIG. 1A, in accordance with some embodiments.

FIG. 1E shows a plan view and a corresponding cross-sectional view of an output region of the microfluidic device of FIG. 1A, in accordance with some embodiments.

FIGS. 2A-2G shows alternative embodiments for a pattern of a first set of piezoelectric actuators in the input region of a microfluidic device, in accordance with some embodiments.

FIG. 3 is a block diagram illustrating example electrical components for a microfluidic device, in accordance with some embodiments.

FIG. 4 is a flow diagram illustrating an example method of dissociating and manipulating cells or particles in a microfluidic channel, in accordance with some embodiments.

In accordance with common practice, the various features illustrated in the drawings are not necessarily drawn to scale, and like reference numerals can be used to denote like features throughout the specification and figures.

### DESCRIPTION OF EMBODIMENTS

Reference will be made to embodiments, examples of which are illustrated in the accompanying drawings. In the following description, numerous specific details are set forth in order to provide a thorough understanding of the various described embodiments. However, it will be apparent to one of ordinary skill in the art that the various described embodiments may be practiced without these particular details. In other instances, methods, procedures, components, circuits, and networks that are well-known to those of ordinary skill in the art are not described in detail so as not to unnecessarily obscure aspects of the embodiments.

The microfluidic devices described herein allow for electrical and/or optical sensing of one or more cells (or other particles such as bacteria, viruses, elements, compounds, and the like). The microfluidic aspect of the devices allows for precise dissociating and manipulating particles in a microfluidic channel (e.g., using sets of piezoelectric actuators) and accurate/precise measurements of particle properties. Additionally, the microfluidic device having an outlet port (e.g., a nozzle) with corresponding piezoelectric actuators allows for direct ejection (e.g., jetting) of cells (e.g., after they have been processed) and regulation of the flow rate through the microfluidic device.

The microfluidic devices described herein can improve design flexibility, addressing a broader design space utilizing the piezoelectric material placement. For example, small (e.g., less than 20 micron (μm)) to large (e.g., greater than 200 μm) piezoelectric structures can be implemented and used, which provide a wider range of performance attributes. Additionally, the piezoelectric membranes described herein can improve processing (e.g., case of processing and reproducibility). For example, the piezoelectric membrane can be placed between a silicon on insulator (SOI) handler layer and a glass top layer and encased in a passivation material (e.g., a dielectric material) such that the membrane is not exposed to mechanical stress. For example, the membrane can be formed by selectively etching the SOI. This can also allow for easier wafer handling. The devices described herein can also improve packaging flexibility and/or improve robustness (improved yield) by mechanically protecting the piezoelectric actuators and membranes.

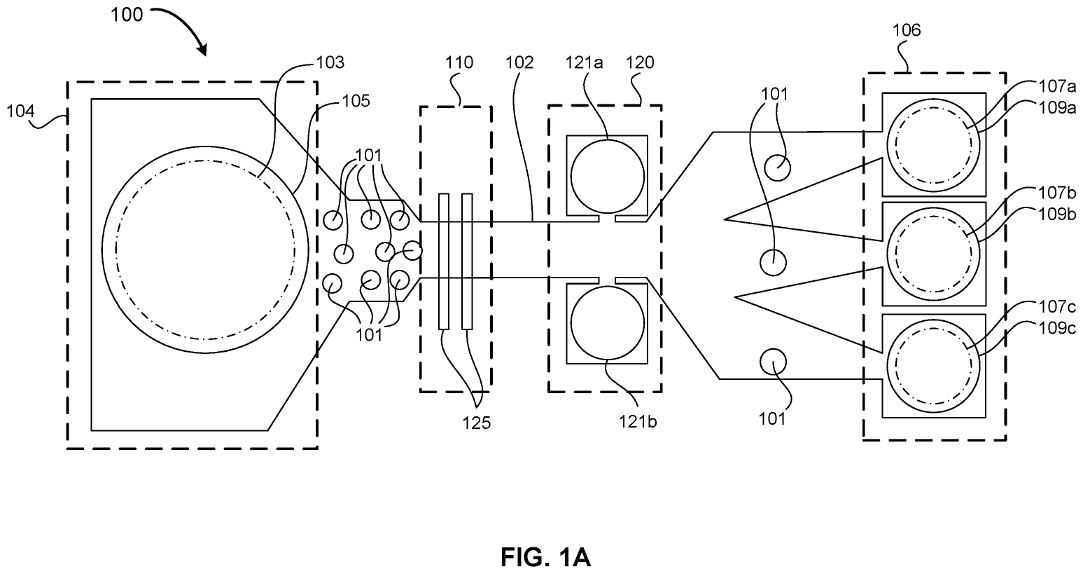

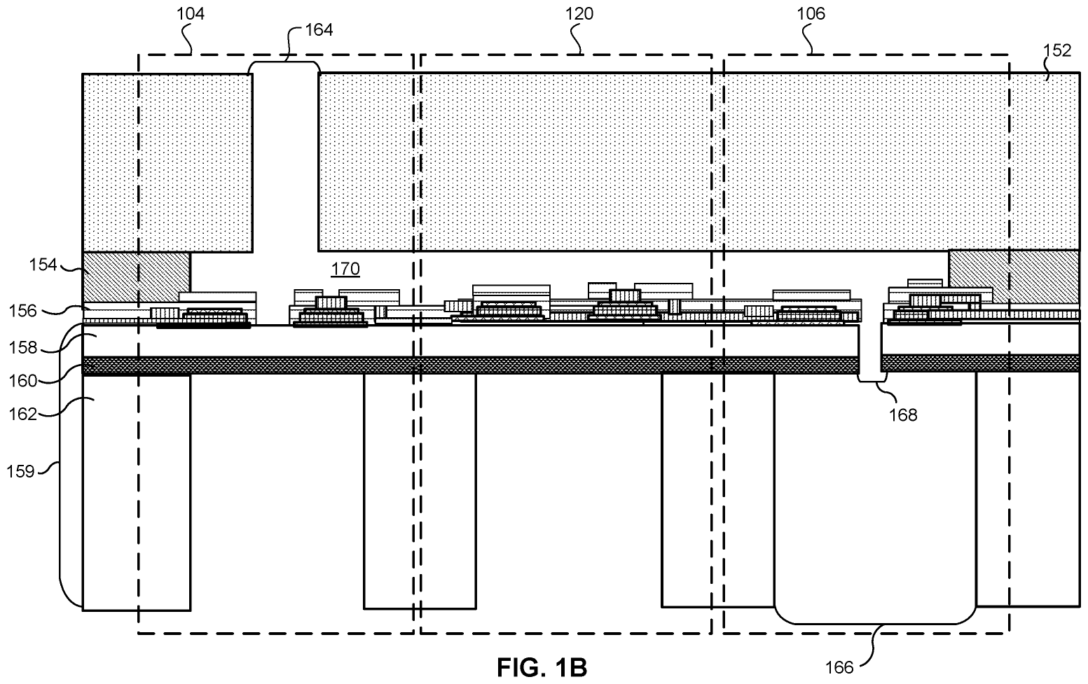

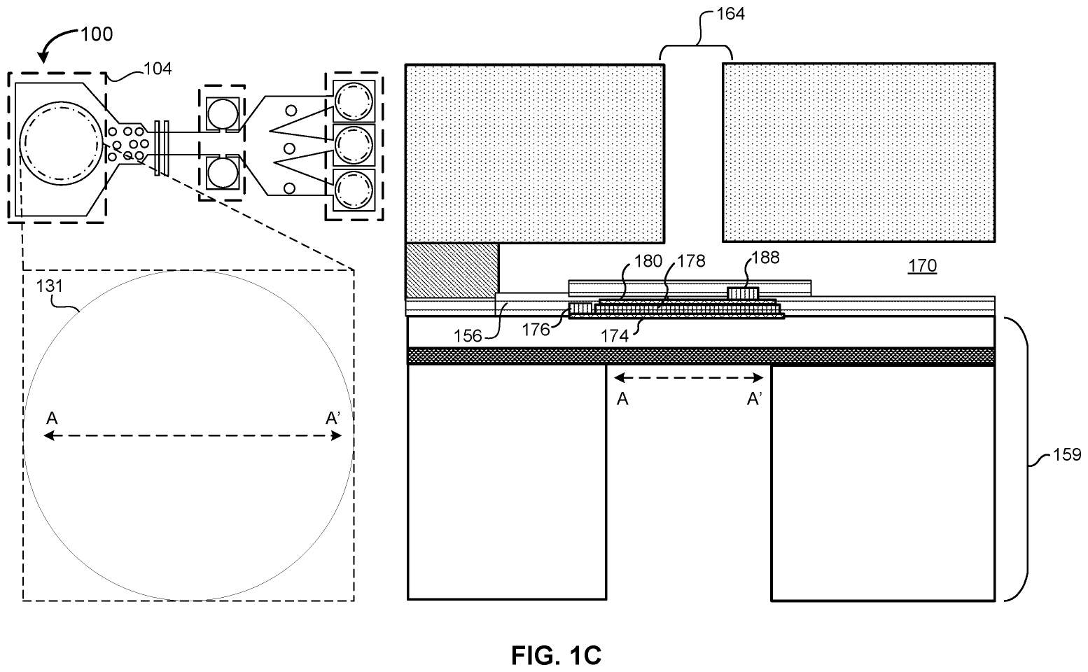

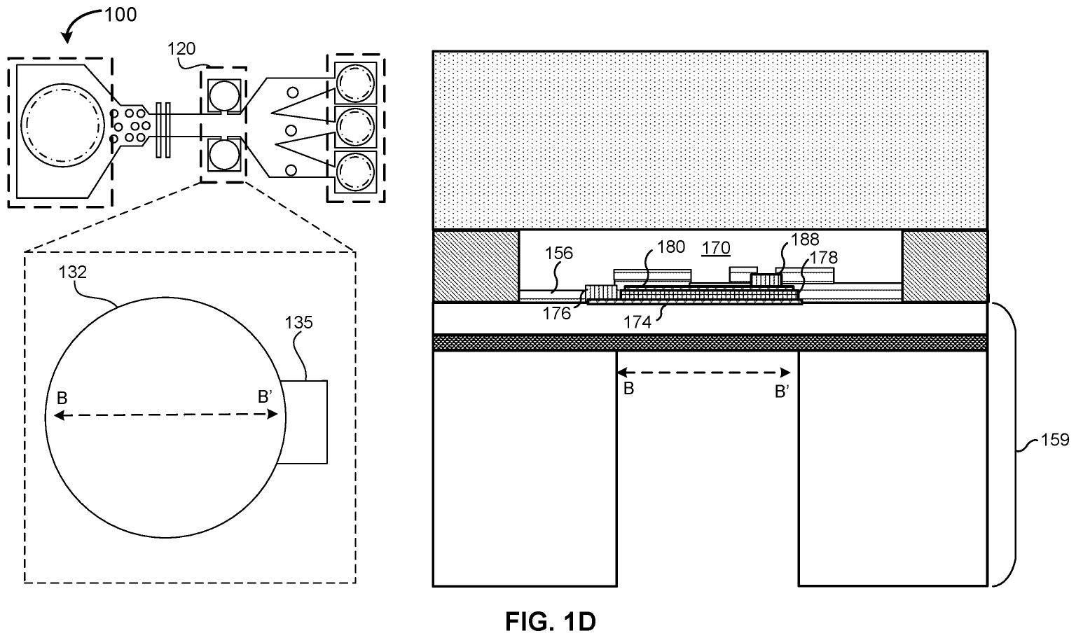

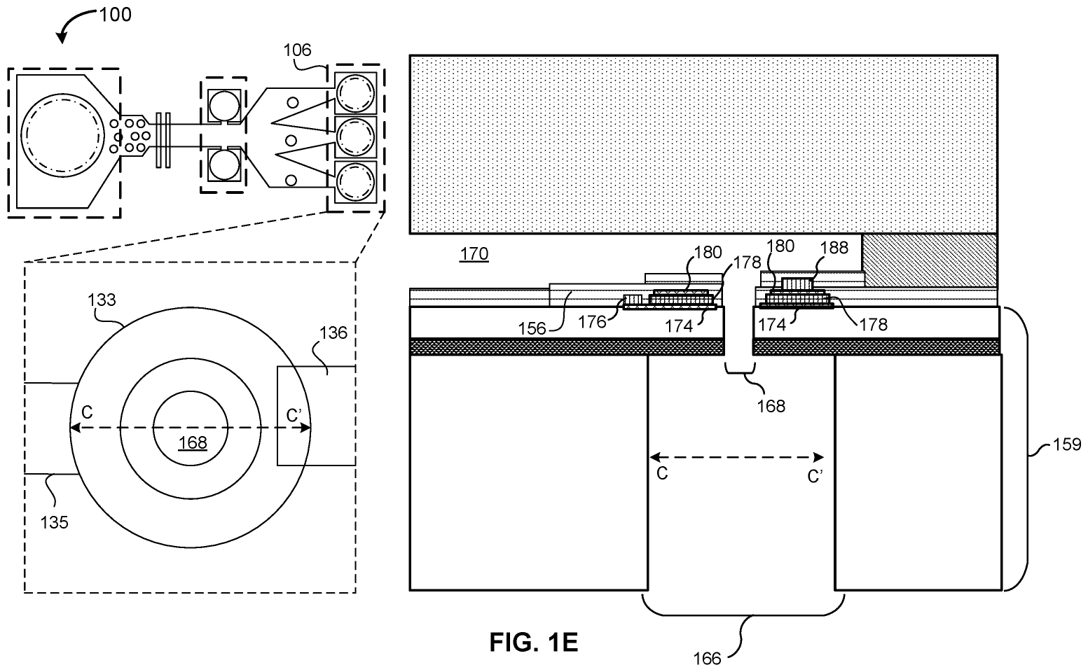

FIG. 1A shows a plan view of a microfluidic device 100 in accordance with some embodiments. The microfluidic device 100 includes a fluid channel 102 (e.g., a microfluidic channel) formed on a substrate. In some embodiments, the fluid channel 102 is formed by coupling a first substrate with an indentation, recess, or notch with a second substrate so that the fluid channel 102 is defined between the first substrate and the second substrate. The fluid channel 102 has an inlet 103 and a plurality of outlets 107a-107c (e.g., three outlets as illustrated in FIG. 1A). The locations of the inlet 103 and the plurality of outlets 107a-107c shown with respect to the fluid channel 102 in FIG. 1A are examples. The inlet 103 and the plurality of outlets 107a-107c may be defined at other locations along the length dimension of the fluid channel 102 or the microfluidic device 100.

In some embodiments, the microfluidic device 100 comprises more or less outlets than shown in FIG. 1A (e.g., 1, 2, 4, 5, or 6 outlets). In some embodiments, a length of the fluid channel 102 (e.g., measured from the inlet 103 to the plurality of outlets 107a-107c) is in the range of 1 millimeter (mm) to 50 mm (e.g., 15 mm). In some embodiments, a width of the microfluidic device 100 is in the range of 0.2 mm to 5 mm (e.g., 0.7 mm). In some embodiments, a width of the fluid channel 102 is configured based on a size of a particle to be analyzed. For example, for cellular measurements, the width of the fluid channel 102 may be configured in accordance with the size of a cell such that only a single cell is detected at a time (e.g., a width of the fluidic channel 102 at a sensing region 110). In some embodiments, the fluid channel 102 includes one or more portions that have different respective widths. For example, the fluid channel 102 may include portions having (protruding) shapes such that widths of the portions are greater than the width fluid channel 102 at the sensing region 110 (e.g., a width of the fluidic channel 102 at a regulation region 120). Similarly, the fluid channel 102 may include one or more portions with widths narrower than the width of the fluidic channel 102 at the sensing region 110. In some embodiments, a wider fluid channel 102 results in a slower velocity of particles flowing in the corresponding portion of the fluid channel 102 (e.g., when the fluid channel 102 has a uniform height). As such, for example, a wider portion may be used to reduce the velocity of the particles (e.g., immobilize the particles), which can allow for more time for analyzing the particles.

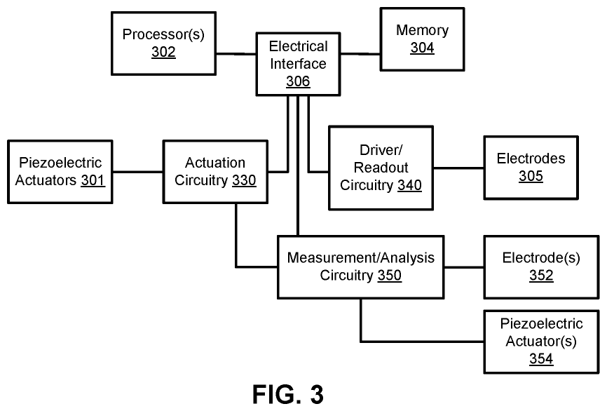

The device 100 also includes an input region 104 (also sometimes referred to as an inlet region) for receiving a sample fluid with particles (e.g., cells) as an input to the microfluidic device 100 and providing the sample fluid from the inlet port to the fluid channel 102 via the inlet 103. In accordance with some embodiments, microfluidic device 100 includes a plurality of pillars 101 adjacent to (or within) the input region 104. The pillars 101 are shaped and arranged to disrupt (e.g., separate) the particles in the sample fluid. The shape and size of the input region 104 in FIG. 1A is a mere example. The microfluidic device 100 includes a first set of piezoelectric actuators 105 at the input region 104 (e.g., around the inlet 103). In some embodiments, the first set of piezoelectric actuators 105 is a single piezoelectric actuator (e.g., as illustrated in FIG. 1A). In some embodiments, the first set of piezoelectric actuators 105 is located adjacent to the inlet 103 and is configured to induce a laminar flow from the inlet 103 toward the plurality of outlets 107a-107c. In some embodiments, the first set of piezoelectric actuators 105 is configured to generate inertial and acoustic turbulence in asymmetric channels to cause chaotic advection and drive localized non-linear behavior in a laminar flow field. In some embodiments, upon application of an electrical signal from actuation circuitry (e.g., actuation circuitry 330 described with respect to FIG. 3), the first set of piezoelectric actuators 105 generates oscillations that create displacement as well as acoustic waves which causes mixing and dissociation of the sample fluid and controls localized inertial movement of the particles to induce a laminar flow in the fluid channel 102. In some embodiments, the first set of piezoelectric actuators 105 is configured to adjust particle mixing, particle dissociation, particle agglomeration, particle coagulation, particle clogging in the fluid sample (e.g., to dissociate any clogged particles and/or prevent clogging of the input region 104 and/or a sensing region 110). In some embodiments, the first set of piezoelectric actuators 105 is configured to adjust particle and/or fluidic viscosity and/or density of the fluid sample. In some embodiments, the first set of piezoelectric actuators 105 is configured to control and/or prevent bubble formation in the fluid sample.

The microfluidic device 100 further includes an output region 106 for collecting at least a portion of the sample fluid from the fluid channel 102 and ejecting or delivering the sample fluid portion via the plurality of outlets (e.g., nozzles) 107a-107c for disposal or further processing or analysis. In some embodiments, the output region 106 includes a plurality of output subregions, each subregion including one of the plurality of outlets 107a-107c. The shape and size of the output region 106 and the output subregions in FIG. 1A are mere examples. The output region 106 includes a third set of piezoelectric actuators 109a-109c, located adjacent to each of the plurality of outlets 107a-107c for ejecting a portion of the fluid in the fluid channel 102. In some embodiments, each output subregion includes a subset of the third set of piezoelectric actuators 109a-109c, each subset adjacent to a respective outlet of the plurality of outlets 107a-107c. As such, different portions of the sample fluid may be collected at, and ejected from, a corresponding output subregion. In some embodiments, the microfluidic device 100 is configured such that each output subregion, and each outlet of the plurality of outlets 107a-107c, is associated with a type of particle and/or a quality of the particles in the fluid sample. For example, the regulation region 120 is configured to cause different types of particles in the fluid sample to flow to different outlets of the plurality of outlets 107a-107c.

The microfluidic device 100 further includes the regulation region 120 for regulating the flow of the sample fluid and manipulating the particles (e.g., levitating, rotating, localizing, and/or deflecting a cell) in the sample fluid. The shape and size of the regulation region 120 in FIG. 1A is a mere example. The regulation region 120 includes a second set of piezoelectric actuators 121a-121b. In some embodiments, the second set of piezoelectric actuators 121a-121b is located between the inlet 103 and the plurality of outlets 107a-107c (e.g., the inlet 103 may be located in an upstream region of the fluid channel 102, the plurality of outlets 107a-107c may be located in a downstream region of the fluid channel 102, and the second set of piezoelectric actuators may be located in a midstream region of the fluid channel 102). In some embodiments, the second set of piezoelectric actuators 121a-121b is located between the sensing region 110 and the output region 106. In some embodiments, the second set of piezoelectric actuators 121a-121b include actuators arranged on opposite sides of the fluid channel 102 (e.g., the two actuators illustrated in FIG. 1A). In some embodiments, the second set of piezoelectric actuators 121a-121b are configured to deflect the particles in the fluid sample to a specified output subregion of the output region 106 based on one or more properties of the particle in the fluid sample (e.g., each output subregion corresponding to a particular cell or a type of cell). The manipulation of the particles of the sample fluid may be achieved, for example, by the oscillations and displacement caused by activation of the second set of piezoelectric actuators 121a-121b. For example, the oscillations and displacement provided by the second set of piezoelectric actuators 121a-121b may control movement, position, rotation and/or acceleration of the particles in the fluid channel 102. In some embodiments, the second set of piezoelectric actuators 121a-121b are configured to vibrate in a direction that is perpendicular to the flow of the sample fluid in the fluid channel 102. For example, two actuators placed on either side of the fluid channel 102 operate in tandem to either deflect the particles downstream into one of the output subregions and/or regulate the location of the particles (e.g., sorting may be performed based on label-free phenotypic analysis of cells and various cell population types can be isolated and/or enriched). The manipulation of the particles may be based on instructions from the sensing region 110 and/or the actuation circuitry 330. In some embodiments, the second set of piezoelectric actuators include more than two piezoelectric actuators (e.g., two pairs, three pairs, or four pairs of piezoelectric actuators). In some embodiments, piezoelectric actuators of the second set of piezoelectric actuators are offset from one another (e.g., the piezoelectric actuator 121a is positioned upstream from the piezoelectric actuator 121b, or vice versa). In some embodiments, piezoelectric actuators of the second set of piezoelectric actuators have differing sizes and/or shapes.

In some embodiments, each of the three sets of piezoelectric actuators (the first set of piezoelectric actuators 105, the second set of piezoelectric actuators 121a-121c, and the third set of piezoelectric actuators 109a-109c) includes one or more piezoelectric actuators (e.g., a piezo micro-electro-mechanical system (MEMS) actuator). In some embodiments, each of the three sets of piezoelectric actuators includes two or more piezoelectric actuators. In some embodiments, the first set of piezoelectric actuators 105 comprise one or more actuators having a first size, the second set of piezoelectric actuators 121a-121b comprise one or more actuators having a second size, different than the first size, and the third set of piezoelectric actuators 109a-109c comprise one or more actuators having a third size, different from the first size and the second size (e.g., the first size is three times the second size and two times the third size). In some embodiments, each of the three sets of piezoelectric actuators have a thickness in a range of 0.1 μm to 5 μm. In some embodiments, each of the three sets of piezoelectric actuators is arranged on a membrane (e.g., the membrane is composed of a substrate with a thickness in a range of 1 μm to 5 μm).

In some embodiments, the microfluidic device 100 includes actuation circuitry electrically coupled to each of the three sets of piezoelectric actuators (e.g., actuation circuitry 330 described with respect to FIG. 3). In some embodiments, each of the three sets of piezoelectric actuators is configured to vibrate at a configurable frequency (e.g., in a range of 1 kilohertz (kHz) to 100 gigahertz (GHz)). In some embodiments, operating in the frequency range comprises vibrating in the frequency range. In some embodiments, at least a subset of the three sets piezoelectric actuators described herein are configured to use an operating frequency within the frequency range that is based on a type of particles in the sample fluid and/or an intended operation (e.g., dissociation, levitation, rotation, and/or mixing). As an example, a megahertz (MHz) subrange may be used to levitate particles (e.g., cells), a kHz and/or MHz subrange may be used to rotate particles, and a kHz range may be used to sort particles. As another example, the first set of piezoelectric actuators may operate in a kHz-MHz range to perform ultrasonication of the sample, where the selected frequency may depend on the desired function (e.g., disrupt clumps, disrupt clogs and/or push/stimulate fluid flow) and the types of particles in the sample.

In some situations, the piezoelectric actuators disclosed herein operate at low power and their operating mode is adjustable based on the performance function desired. In some embodiments, each of the three sets of piezoelectric actuators is configured to have an operating voltage in the range of 0.1 volt (V) to 100 V (e.g., a range of 0.1 V to 30 V) based on a type of actuator and/or a type of sample. For example, a PZT actuator may have an operating voltage up to 30 V whereas a polymer actuator may have an operating voltage in the range up to 50 V. In some embodiments, each of the three sets of piezoelectric actuators are configured to have a deflection in a range of 5 nanometers (nm) to 50 μm (e.g., a range 100 nm to 10 μm) based on a type of actuator and/or a type of sample. In some embodiments, upon application of an electrical signal from the actuation circuitry, each of the three sets of piezoelectric actuators (either individually or synchronously) generate oscillations that create displacement as well as acoustic waves, which control localized inertial movement of the particles in the fluid channel 102 in the three-dimensional x, y, and z planes with sub-μm level control. In some embodiments, each of the three sets of piezoelectric actuators induce a laminar flow from the inlet 103 toward the plurality of outlets 107a-107c. In some configurations, the sample fluid flows through the fluid channel 102 at a rate between 1 μL/min and 1 mL/min.

In some embodiments, the microfluidic device 100 further includes the sensing region 110 for sensing one or more properties of the particles in fluid sample. In some embodiments, the sensing region 110 overlaps with (e.g., is the same as) the regulation region 120. The sensing region 110 comprises a set of electrodes 125 and/or other sensing elements for sensing the one or more properties. In some embodiments, an echo response of the first set of piezoelectric actuators 105 in the input region 104 and/or the second set of piezoelectric actuators 121a-121b in the regulation region 120 is sensed to determine properties of the sample and/or the microfluidic channel (e.g., an actuator's echo response will change if a foreign object is on the actuator). The position of the sensing region 110 in FIG. 1A is a mere example. In some embodiments, the sensing region includes at least one filter (e.g., a filter pillar array). The position of the set of electrodes 125 and the sensing region 110 may be varied along the length of the fluid channel 102. For example, it may be beneficial to have equal distances between each electrode of the set of electrodes to acquire accurate measurements. In some embodiments, the set of electrodes detect electrical signals of particles (e.g., cells) flowing through the fluid channel 102 adjacent to the set of electrodes. In some embodiments, the microfluidic device 100 includes readout circuitry (e.g., driver/readout circuitry 340 described with respect to FIG. 3) electrically coupled with the set of electrodes. In some embodiments, the readout circuitry receives electrical signals from the set of electrodes and relays the electrical signals (with or without processing, such as filtering) to one or more processors of, or operationally connected with, the microfluidic device 100.

In some embodiments, the set of electrodes charge particles flowing through the fluid channel 102 by applying an electrical field to the sample fluid to charge the particles such that the particles may be manipulated by another electrical field. In some embodiments, the distance between a pair of the electrodes is configured such that only a single cell is charged and/or manipulated at a time. In some embodiments, the microfluidic device 100 includes driver circuitry (e.g., driver circuitry 340 described with respect to FIG. 3) electrically coupled to the set of electrodes. In some embodiments, the driver circuitry is configured to produce electrical signals in the MHz and GHz frequency domains. In some embodiments, the frequency of the electrical signals provided to the electrodes depends on a type or types of the particles to be analyzed using the microfluidic device 100. In some embodiments, the driver circuitry is configured to produce electrical signals with a voltage in the range of 1 V to 100 V. In some embodiments, the driver circuitry is configured to produce electrical signals with a pulse in a range from 1 μs to 20 μs. In some embodiments, the pulse is a sawtooth pulse, a square pulse, or a sinusoidal pulse.

In some embodiments, each particle may pass the vicinity of one or more pairs of electrodes for a period between 0.1 milliseconds (ms) and 100 ms. In some embodiments, each particle may pass the vicinity of one or more second pairs of electrodes for a period between 0.1 ms and 100 ms. In some embodiments, a separation distance between a pair of electrodes as well as a distance between the first electrodes and the second electrodes are configured based on a type or types of the particles to be analyzed using the device 100. In some embodiments, a particle processing rate in the microfluidic device 100 is between from 100 particles per minute and 1 million particles per minute.

In some embodiments, the set of electrodes is located on a same substrate as one another and/or the piezoelectric components. In some embodiments, a pair of electrodes is located on different substrates (e.g., one electrode of a pair of electrodes is located on a bottom substrate and another electrode of the pair of electrodes is located on a top substrate). In some embodiments, the electrodes have a thickness in the range of 0.01 μm-2 μm. In some embodiments, the electrodes are composed of gold (Au) and/or platinum (Pt). In some embodiments, the additional set of electrodes are shielded from each of the three sets of piezoelectric actuators. For example, the electrodes are on a first board/chip and each of the sets of piezoelectric actuators are on a separate board/chip. In some embodiments, the operation of the electrodes is based on the operation of each of the sets of piezoelectric actuators (e.g., the electrode operation is timed to reduce/minimize interference from operation of each of the sets of piezoelectric actuators).

FIG. 1B shows a cross-section view of the microfluidic device 100, in accordance with some embodiments. As shown in FIG. 1B, the microfluidic device 100 includes an optical layer 152 (e.g., composed of a transparent or translucent material such as glass) with an inlet channel 164 (e.g., the inlet 103 shown in FIG. 1A). The optical layer 152 is coupled to a substrate 159 via a polymer layer 154. The polymer layer 154 defines a fluidic channel 170 (e.g., the fluid channel 102 shown in FIG. 1A). In some embodiments, the fluidic channel 170 is a microfluidic channel. In accordance with some embodiments, an outlet channel 166 is defined in the substrate 159 (e.g., etched in the handler layer 162). In some embodiments, the three sets of piezoelectric actuators described in reference to FIG. 1A are arranged over portions of the substrate from which the silicon base layer have been removed (e.g., to form a membrane below the actuators) (e.g., as illustrated in FIGS. 1B-1E). In the example of FIG. 1B, the substrate 159 includes a handler layer 162 (e.g., composed of silicon), a buried oxide (BOX) layer 160 (e.g., composed of SiO2), and a device layer 158. In accordance with some embodiments, an outlet 168 (e.g., one outlet of the plurality of outlets 107a-107c shown in FIG. 1A) is defined in the substrate 159 (e.g., through a BOX layer 160 and the device layer 158). A passivation layer 156 (e.g., an encapsulation layer) (e.g., composed of nitride, silicon nitride, silicon carbide, SiO2, aluminum nitride, aluminum oxide, and/or a photo-imagable polymer) is coupled to the substrate 159. In accordance with some embodiments, electrodes are arranged in the fluidic channel 170. In some embodiments, the device layer 158 has a thickness in the range of 1 μm to 5 μm. In some embodiments, the handler layer 162 has a thickness in the range of 200 μm to 600 μm. In some embodiments, the passivation layer 156 has a thickness in the range of 0.01 μm to 1 μm.

In some embodiments, the fluidic channel 170 is defined by the optical layer 152, the polymer layer 154, and the device layer 158. In some embodiments, the channel 170 has a height between 10 μm and 1 mm (e.g., 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, or 1 mm, or within a range between any two of the aforementioned values). In some embodiments, the optical layer 152 has a thickness between 5 μm and 2 mm (e.g., 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 mm, 1.1 mm, 1.2 mm, 1.3 mm, 1.4 mm, 1.5 mm, 1.6 mm, 1.7 mm, 1.8 mm, 1.9 mm, or 2 mm, or within a range between any two of the aforementioned values). In some embodiments, the substrate 159 is 500 μm thick. In some embodiments, the inlet channel 164 is defined in the substrate 159 (e.g., in addition to, or alternatively to, being defined in the optical layer 152).

In some embodiments, the polymer layer 154 is positioned between optical layer 152 and the substrate 159 (e.g., between the optical layer 152 and the device layer 158). In some embodiments, the polymer layer 154 is composed of polyvinylidene fluoride (PVDF) and its copolymers, polyamides, and paralyne-C, polyimide and polyvinylidene chloride (PVDC), and diphenylalanine peptide nanotubes (PNTs). In some embodiments, the polymer layer 154 is adapted and/or positioned to adhere the optical layer 152 (e.g., a first substrate) and the substrate 159 (e.g., a second substrate) to one another. For example, if the polymer layer 154 is not included, then the optical layer 152 may not bond to the substrate 159. In some embodiments, the polymer layer 154 is composed of a photo-imagable material. For example, imaging the polymer layer 154 provides definition of the fluidic channel 170, such as its width, height, and curvature (e.g., which can improve the signal-to-noise ratio (SNR) for single cell sensing). In some embodiments, the polymer layer 154 is adapted and/or positioned to provide stress relief for the microfluidic device 100 (e.g., to prevent stress cracking when the chip is assembled in a package). In some embodiments, the polymer layer 154 is cured/hardened (e.g., submitted to multiple stages of curing/hardening). In some embodiments, the polymer layer 154 is submitted to a temperature that exceeds a transition temperature (e.g., 150 degrees Celsius) for the polymer layer 154, whereby the polymer layer 154 cures and bonds the optical layer 152 (e.g., glass) to the substrate 159 (e.g., silicon). In some embodiments, the polymer layer 154 is composed of a liquid or a dry film. The polymer layer 154 may be a negative or positive photo-resist. In some embodiments, the polymer layer 154 is composed of an epoxy (e.g., bisphenol-A) and/or polyimides with photo initiators (e.g., added to drive cross linking based on the wavelength of light).

FIGS. 1C, 1D, and 1E show cross-sectional views of the microfluidic device 100 at each of the input region 104, the regulation region 120, and one subregion of the output region 106, respectively, in accordance with some embodiments. FIG. 1C shows the input region 104 with the inlet channel 164 which, in some embodiments, is defined in the optical layer 152. The input region 104 further includes the first set of piezoelectric actuators 105 situated below the inlet channel 164. FIG. 1D shows the regulation region 120 with one of the second set of piezoelectric actuators 121a-121b. FIG. 1E shows the one subregion of the output region 106 with the outlet channel 166 and outlet 168 (e.g., one of the plurality of outlets 107a-107c) which, in some embodiments, is defined in the substrate 159. The one subregion further includes one subset of the third set of piezoelectric actuators 109a-109c situated around the outlet 168.

Each of the first set of piezoelectric actuators 105, the second set of piezoelectric actuators 121a-121b, and the third set of piezoelectric actuators 109a-109c includes a bottom electrode 174, top electrode 180, and piezoelectric layer 178. Each of the piezoelectric actuators further includes a bottom contact 176 coupled to the bottom electrode 174 and a top contact 188 coupled to the top electrode 180. In some embodiments, the bottom electrode 174 and/or the top electrode 180 are each composed of an electrically conductive material (e.g., copper, aluminum, gold, or platinum), Strontium oxide (SRO), and/or Titanium. In some embodiments, the bottom electrode 174 and/or the top electrode 180 has a thickness in the range of 10 nm to 50 nm.

In some embodiments, a first conductive layer is coupled to the top contact 188 and a second conductive layer is coupled to the bottom contact 176. For example, the first and second conductive layers comprise conductive routing configured to supply electrical signals (e.g., actuation signals) to the piezoelectric actuators. In some embodiments, the first and second conductive layers couple the respective piezoelectric actuator to control circuitry (e.g., the actuation circuitry 330). In some embodiments, the conductive layers are each composed of an electrically conductive material (e.g., copper, aluminum, gold, or platinum). In some embodiments, a respective bond pad is coupled to each of the conductive layers (e.g., the respective bond pads provide off-chip electrical coupling to the piezoelectric actuators via the conductive layers). The passivation layer 156 separates the piezoelectric layer 178 and the electrodes 174 and 180 from the fluidic channel 170. In some embodiments, an intermediate adhesion layer is arranged between the piezoelectric layer 178 and the passivation layer 156. In some embodiments, the intermediate adhesion layer is configured to match a mechanical impedance between the piezoelectric layer 178 and the passivation layer 156. In some embodiments, the intermediate adhesion layer is composed of metal (e.g., Titanium (Ti)). In some embodiments, the intermediate adhesion layer is configured to reduce cracking in the passivation layer 156. In some embodiments, the intermediate adhesion layer has a thickness in the range of 10 nm-300 nm.

In some embodiments, the piezoelectric layer 178 has a thickness between 0.1 μm and 100 μm (e.g., 0.1 μm, 0.5 μm, 1 μm, 2 μm, 5 μm, 10 μm, 20 μm, 30 μm, 40 μm, 50 μm, 60 μm, 70 μm, 80 μm, 90 μm, or 100 μm, or within a range between any two of the aforementioned values). In some embodiments, the piezoelectric layer 178 is positioned on a silicon-on-insulator (SOI) layer. In some embodiments, the silicon-on-insulator (SOI) layer is connected to one or more of the contacts (e.g., the contact 176). In some embodiments, the piezoelectric layer 178 is composed of PZT and has a thickness in the range of 0.1 μm-10 μm (e.g., 2 μm).

FIG. 1C additionally shows a plan view of a first piezoelectric membrane 131, in accordance with some embodiments. The first piezoelectric membrane 131 is positioned in the input region 104. The first piezoelectric membrane 131 may include the substrate 159 (e.g., a buried oxide layer and/or a device layer), the passivation layer 156, a piezoelectric material 178 (e.g., PZT), and associated conductive layers (e.g., electrodes, vias, contacts, and/or routing). For example, the cross-sectional view in FIG. 1C illustrates the first piezoelectric membrane 131 along the A-A′ diameter.

FIG. 1D additionally shows a plan view of a second piezoelectric membrane 132, in accordance with some embodiments. The second piezoelectric membrane 132 is positioned in the regulation region 120. The second piezoelectric membrane 132 may include the substrate 159, the passivation layer 156, the piezoelectric material 178, and associated conductive layers (e.g., electrodes, vias, contacts, and/or routing). In some embodiments, the second piezoelectric membrane 132 includes a first contact 135 (e.g., the top contact 188) electrically coupled to a respective electrode (e.g., the top electrode 180). The first electrode may be coupled to one or more piezoelectric layers to actuate the piezoelectric material and regulating the flow of the sample fluid and manipulating the particles in the sample fluid. For example, the cross-sectional view in FIG. 1D illustrates the second piezoelectric membrane 132 along the B-B′ diameter.

FIG. 1E additionally shows a plan view of a third piezoelectric membrane 133, in accordance with some embodiments. The third piezoelectric membrane 133 is positioned in the output region 106. The third piezoelectric membrane 133 may include the substrate 159, the passivation layer 156, the piezoelectric material 178, and associated conductive layers (e.g., electrodes, vias, contacts, and/or routing). In some embodiments, the third piezoelectric membrane 133 includes the first contact 135 (e.g., the bottom contact 176) electrically coupled to the respective electrode (e.g., the bottom electrode 174), a second contact 136 (e.g., the top contact 188) electrically coupled to a second respective electrode (e.g., the top electrode 180), and the outlet 168 (e.g., one of the plurality of outlets 107a-107c) (e.g., a nozzle). The first and second electrodes may be coupled to one or more piezoelectric layers to actuate the piezoelectric material and eject the sample fluid via the outlet 137. In some embodiments, the outlet 168 has a diameter of less than 200 μm (e.g., 30 μm, 60 μm, or 120 μm). For example, the cross-sectional view in FIG. 1E illustrates the third piezoelectric membrane 133 along the C-C′ diameter.

In some embodiments, the first piezoelectric material is polyvinylidene fluoride, gallium phosphate, sodium bismuth titanate, lead zirconate titanate, quartz, berlinite (AlPO4), sucrose (table sugar), rochelle salt, topaz, tourmaline-group minerals, lead titanate (PbTiO3), langasite (La3Ga5SiO14), gallium orthophosphate (GaPO4), lithium niobate (LiNbO3), lithium tantalate (LiTaO3), any of a family of ceramics with perovskite, tungsten-bronze, potassium niobate (KNbO3), sodium tungstate (Na2WO3), Ba2NaNb5O5, Pb2KNb5O15, sodium potassium niobate ((K,Na)NbO3) (e.g., NKN, or KNN), bismuth ferrite (BiFcO3), sodium niobate (NaNbO3), barium titanate (BaTiO3), bismuth titanate (Bi4Ti3O12), sodium bismuth titanate (NaBi(TiO3)2), zincblende crystal, GaN, InN, AlN, and ZnO. In some embodiments, the piezoelectric material is of any of BKT-BMT-BFO set. For example, KNN such as (K0.5Na0.5)NbO3, BKT such as (Bi0.5K0.5)TiO3, BMT such as Bi(Mg0.5Ti0.5)O3, BFO such as BiFeO3, BNT such as (Bi0.5Na0.5)TiO3, and/or BT such as BaTiO3.

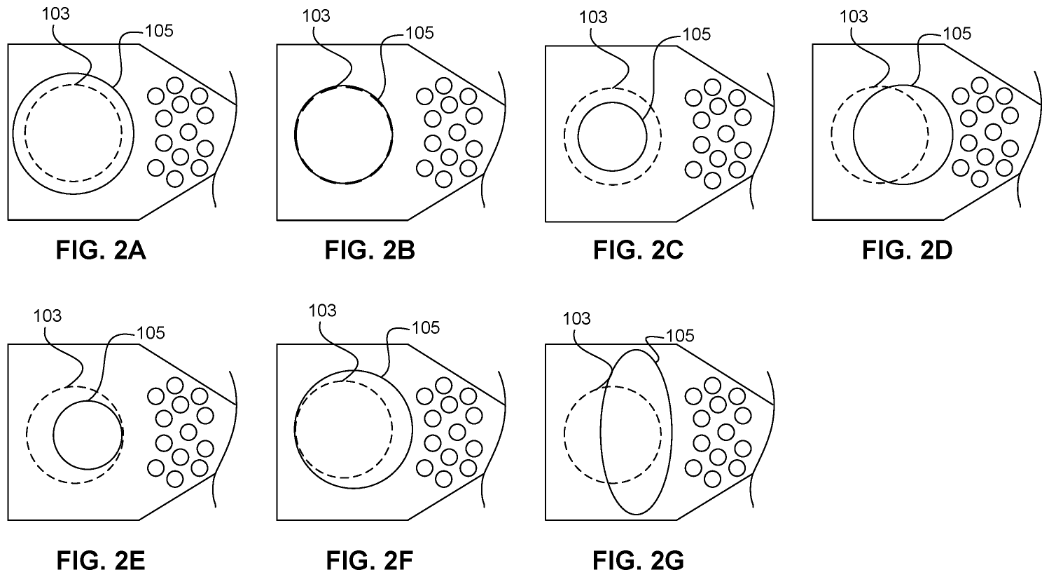

FIGS. 2A-2G illustrate alternative embodiments for a pattern of the first set of piezoelectric actuators 105 in the input region 104, in accordance with some embodiments. FIG. 2A illustrates a first configuration in which the first set of piezoelectric actuators 105 is aligned with the inlet 103 and has a larger diameter than a diameter the inlet 103 (e.g., as illustrated in FIG. 1A), in accordance with some embodiments. FIG. 2B illustrates a second configuration wherein the first set of piezoelectric actuators 105 is aligned with the inlet 103 and has a same diameter as the diameter of the inlet 103, in accordance with some embodiments. FIG. 2C illustrates a third configuration wherein the first set of piezoelectric actuators 105 is aligned with the inlet 103 and has a smaller diameter than the diameter the inlet 103. FIG. 2D illustrates a fourth configuration wherein the first set of piezoelectric actuators 105 is offset from the inlet 103 (e.g., a center the first set of piezoelectric actuators 105 is closer to the regulation region 120 relative to the inlet 103, as illustrated in FIG. 2D) and has a same diameter as the diameter of the inlet 103. FIG. 2E illustrates a fifth configuration wherein the first set of piezoelectric actuators 105 is offset from the inlet 103 (e.g., the center the first set of piezoelectric actuators 105 is closer to the regulation region 120 relative to the inlet 103, as illustrated in FIG. 2E) and has a smaller diameter as the diameter of the inlet 103. FIG. 2F illustrates a sixth configuration wherein the first set of piezoelectric actuators 105 is offset from the inlet 103 (e.g., the center the first set of piezoelectric actuators 105 is closer to the regulation region 120 relative to the inlet 103, as illustrated in FIG. 2F) and has a larger diameter as the diameter of the inlet 103. FIG. 2G illustrates a seventh configuration wherein the first set of piezoelectric actuators 105 is offset from the inlet 103 (e.g., the center the first set of piezoelectric actuators 105 is closer to the regulation region 120 relative to the inlet 103, as illustrated in FIG. 2G) and is ovular.

In accordance with some embodiments, a pattern of the inlet 103 and/or the pattern of the first set of piezoelectric actuators 105 are different shapes (e.g., circular, ovular, rectangular, etc.). In accordance with some embodiments, the pattern of the inlet 103 and/or the pattern of the first set of piezoelectric actuators 105 are chosen based on a desired function of the input region 104, a quality of the sample fluid, and/or a quality of the particles. For example, a pattern and/or a relative location of the inlet 103 and/or the first set of piezoelectric actuators may be chosen to maximize dissociation of the particles of the fluid sample, achieve a desired laminar flow rate in the fluid channel 102, and/or prevent bubble formation in the fluid sample, as desired.

FIG. 3 is a block diagram illustrating example electrical components for a microfluidic device in accordance with some embodiments. In some embodiments, a device (e.g., the microfluidic device 100) includes one or more processors 302 (e.g., one or more microcontrollers (MCUs), one or more CPUs, and/or other types of control circuitry) and memory 304. In some embodiments, the memory 304 includes instructions for execution by the one or more processors 302. In some embodiments, the stored instructions include instructions for providing actuation signals to each of the three sets of piezoelectric actuators (e.g., the first set of piezoelectric actuators 105, the second set of piezoelectric actuators 121a-121b, and the third set of piezoelectric actuators 109a-109c). In some embodiments, the actuation signals for the different piezoelectric actuators are configured such that each of the piezoelectric actuators create oscillations at a different frequency. For example, one or more of the piezoelectric actuators may operate at a frequency in the range between 1 kHz and 100 kHz, for example, based on desired flow rates. In some embodiments, the stored instructions include instructions for providing activation signals to the electrodes 305 for charging particles flowing through the fluid channel 102 so that the particles can be manipulated with an electrical field. In some embodiments, the device also includes an electrical interface 306 coupled with the one or more processors 302 and the memory 304. In some embodiments, the device further includes actuation circuitry 330, which is coupled to one or more piezoelectric actuators 301, such as the first set of piezoelectric actuators 105, the second set of piezoelectric actuators 121, and/or the third set of piezoelectric actuators 209a-209c. For example, the actuation circuitry 330 sends electrical signals to the piezoelectric actuators to initiate actuation of the piezoelectric actuators.

In some embodiments, the device further includes driver circuitry 340, which is coupled the electrodes 305. For example, the driver circuitry 340 sends electrical signals to the one or more electrodes to generate an electrical field using the one or more electrodes for charging particles flowing through the fluid channel. In some embodiments, the device further includes readout circuitry (e.g., the driver/readout circuitry 340), which is coupled to one or more electrodes 305 (e.g., the electrodes 125). In some embodiments, the driver/readout circuitry is further coupled to the piezoelectric actuators 301 (e.g., the second set of piezoelectric actuators 121). The readout circuitry receives electrical signals from the one or more electrodes 305 and provides the electrical signals (with or without processing) to the one or more processors 302 via the electrical interface 306.

In some embodiments, the device further includes measurement/analysis circuitry 350 coupled to one or more electrodes 352 (e.g., the electrodes 125) and/or one or more piezoelectric actuator(s) 354 (e.g., any of the piezoelectric actuators described herein). In some embodiments, the measurement/analysis circuitry 350 is configured to detect electrical impedance of particles in the microfluidic channel (e.g., the fluidic channel 102). In some embodiments, the measurement/analysis circuitry 350 is coupled to the actuation circuitry 330 and informs the actuation circuitry 330 how to actuate the one or more piezoelectric actuators 301 (e.g., based on the impedance measurements). In some embodiments, the actuation circuitry 330 is configured to adjust actuation of one or more piezoelectric actuators (e.g., adjust frequency and/or magnitude) based on particle analysis results from the measurement/analysis circuitry 350. For example, if the measurement/analysis circuitry 350 indicate that a clump of particles is present, the one or more piezoelectric actuators 301 are configured to break up the clump. In some embodiments, one or more electrodes are shared between the electrodes 305 and the electrode(s) 352. In some embodiments, one or more piezoelectric actuators are shared between the one or more piezoelectric actuators 301 and the piezoelectric actuator(s) 354.

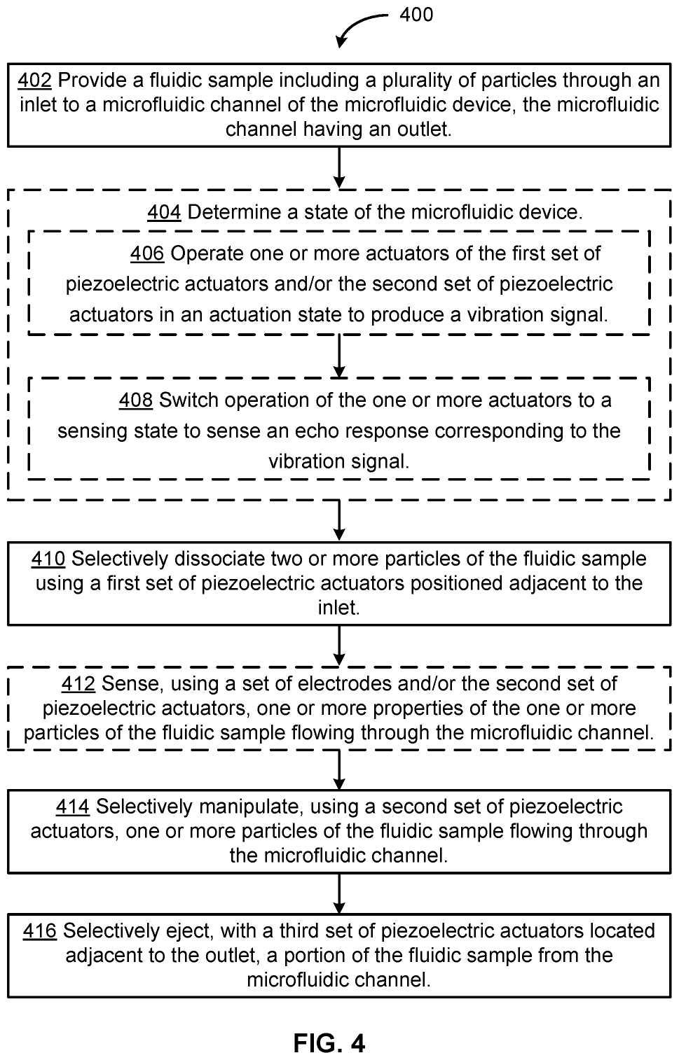

FIG. 4 is a flow diagram illustrating a method 400 of flow control of cells or particles in a microfluidic channel (e.g., the fluid channel 102 and/or the fluidic channel 170) in accordance with some embodiments. In some embodiments, the method 400 is performed at a microfluidic device (e.g., the microfluidic device 100).

The method 400 includes (402) providing a fluidic sample comprising plurality of particles (e.g., cells, molecules, and/or other types of particles) through an inlet (e.g., inlet 103) to a microfluidic channel (e.g., fluid channel 102) of a microfluidic device (e.g., the microfluidic device 100), the microfluidic channel having an outlet (e.g., one of the plurality of outlets 107a-107c). For example, a sample fluid with particles is provided in the fluid channel 102 with the inlet 103 and the plurality of outlets 107a-107c.

In some embodiments, the method 400 further includes (404) determining a state of the microfluidic device. In some embodiments, determining the state of the microfluidic device includes (406) operating one or more actuators of the first set of piezoelectric actuators (e.g., the first set of piezoelectric actuators 105) and/or the second set of piezoelectric actuators (the second set of piezoelectric actuators 121a-121b) in an actuation state to produce a vibration signal. In some embodiments, determining the state of the microfluidic device further includes (408) switching operation of the one or more actuators to a sensing state to sense an echo response corresponding to the vibration signal. For example, if there is a large clump of cells sitting on the one or more actuators, then its response will change. In some embodiments, a first subset of actuators operate in the actuation state to produce one or more vibration signals and a second subset of actuators operate in the sensing state to sense echo responses of the one or more vibration signals. As an example, the acoustic signal received by the second subset of piezoelectric actuators after a rebound indicates a viscosity and/or density of the sample and can be used to assess the agglomeration/coagulation/clogging of the sample.

The method 400 includes (410) selectively dissociating two or more particles of the fluidic sample using a first set of piezoelectric actuators positioned adjacent to the inlet.

In some embodiments, the method 400 includes (412) sensing, using a set of electrodes (e.g., electrodes 352) and/or the second set of piezoelectric actuators (e.g., piezoelectric actuators 354), one or more properties of the one or more particles of the fluidic sample flowing through the microfluidic channel. In some embodiments, the method 400 further includes selectively providing actuation signals (e.g., via the actuation circuitry 330) to the first, second, and third sets of piezoelectric actuators based on obtained sensing data (e.g., via the measurement/analysis circuitry 350). In some embodiments, the actuation signals are generated to create an oscillation frequency in the actuators that is based on a fluid composition (e.g., size and/or concentration of particles) in the microfluidic channel. For example, the third set of actuators may be configured to oscillate such that only 1 cell is ejected at a time. For example, the sense signals may include information about the state of the fluid.

The method 400 includes (414) selectively manipulating, using a second set of piezoelectric actuators, one or more particles of the fluidic sample flowing through the microfluidic channel. In some embodiments, selectively manipulating, using the second set of piezoelectric actuators, the one or more particles of the fluidic sample flowing through the microfluidic channel comprises selectively levitating, rotating, and/or sorting the one or more particles. In some embodiments, selectively manipulating, using the second set of piezoelectric actuators, the one or more particles of the fluidic sample flowing through the microfluidic channel comprises adjusting a frequency of operation of the second set of piezoelectric actuators to perform different types of manipulation.

The method 400 includes (416) selectively ejecting, with a third set of piezoelectric actuators (e.g., the third set of piezoelectric actuators 109a-109c) located adjacent to the outlet, a portion of the fluidic sample from the microfluidic channel. In some embodiments, the third set of piezoelectric actuators and the outlet are sized to be able to eject particles with a diameter ranging from 2 μm-50 μm.

In light of the above disclosure, certain embodiments are described below.

(A1) In one aspect, some embodiments include a microfluidic device (e.g., the microfluidic device 100), including: (i) an inlet channel (e.g., the inlet channel 164), (ii) an outlet channel (e.g., the outlet channel 166), (iii) a microfluidic channel (e.g., the fluid channel 102 and/or the fluidic channel 170), (iv) a first set of piezoelectric actuators (e.g., the first set of piezoelectric actuators 105), (v) a second set of piezoelectric actuators (e.g., the second set of piezoelectric actuators 121a-121b), (vi) a third set of piezoelectric actuators (e.g., the third set of piezoelectric actuators 109a-109c). The microfluidic channel is arranged on a substrate (e.g., the substrate 159) between the inlet channel and the outlet channel such that an outlet (e.g., one of the plurality of outlets 107a-107c and/or the outlet 168) of the microfluidic channel is positioned above at least a portion of the outlet channel. The first set of piezoelectric actuators is arranged adjacent to the inlet channel and configured to dissociate particles (e.g., cells) of a fluidic sample in the microfluidic channel. In some embodiments, the first set of piezoelectric actuators are configured to detach, disconnect, and/or dissociate particles of the fluidic sample from one another. The second set of piezoelectric actuators is arranged between the inlet channel and the outlet channel. The second set of piezoelectric actuators is configured to manipulate the particles of the fluidic sample as the particles move through the microfluidic channel. The third set of piezoelectric actuators is arranged above the outlet channel and is adjacent to the outlet. The third set of piezoelectric actuators is configured to eject a portion of the fluidic sample out of the microfluidic channel via the outlet. In some embodiments, at least some of the piezoelectric actuators described herein are configured to vibrate a configurable frequency.

In some embodiments, the first set of piezoelectric actuators consist of a single actuator positioned under the inlet channel. In some embodiments, the first set of piezoelectric actuators are configured to generate inertial and acoustic turbulence in asymmetric channels to cause chaotic advection and drive localized non-linear behavior in a laminar flow field. In some embodiments, the first set of piezoelectric actuators are configured for ultrasonication of the fluidic sample (e.g., to dissociate any clogged particles and/or prevent clogging of the inlet region and/or the sense region). In some embodiments, the first set of piezoelectric actuators are positioned between the inlet channel and a filter region (e.g., a filter pillar array). In some embodiments, the first set of piezoelectric actuators consist of a single actuator positioned under the inlet channel. In some embodiments, the first set of piezoelectric actuators are configured to generate inertial and acoustic turbulence in asymmetric channels to cause chaotic advection and drive localized non-linear behavior in a laminar flow field. In some embodiments, the first set of piezoelectric actuators is placed asymmetric to the inlet channel and closer to the filter region so as to localize and direct the energy towards the pillars. In some embodiments, the first set of piezoelectric actuators are configurable to selectively dissociate, declog, adjust flow rate, and/or address bubble formation as requested by a controller.

In some embodiments, the second set of piezoelectric actuators include actuators arranged on opposite sides of the microfluidic channel, and the actuators are configured to vibrate in a direction that is perpendicular to the flow of the fluidic sample in the microfluidic channel. In some embodiments, the second set of piezoelectric actuators are configured for levitating, rotating, and/or localizing a particle and/or deflecting a particle to a specified downstream location (e.g., based on instructions from the sensing region 110). For example, actuators placed on either side of the flow channel operate in tandem to either deflect particles into a downstream or regulate the location of the particles. In this way, the sorting may be performed based on label-free phenotypic analysis of cells and various cell population types can be isolated and enriched.

In some embodiments, the third set of piezoelectric actuators include actuators arranged adjacent to the outlet channel (or a plurality of outlet channels). In some embodiments, the third set of piezoelectric actuators are configured for ejecting and/or delivering the sample fluid portion via the outlet channel (or the plurality of outlet channels).

(A2) In some embodiments of A1, at least one of the first set of piezoelectric actuators, the second set of piezoelectric actuators, and the third set of piezoelectric actuators comprises one or more micro-electro-mechanical system (MEMS) actuators. For example, the microfluidic device may include a set of MEMS-fabricated vibrating piezo electrical devices operating in the KHz to GHz range, positioned at various locations in a laminar flow field, configured to create inertial and acoustic perturbations, either individually or in synchronicity with other piezo devices to mix, unclog, levitate, and/or rotate cells and/or particles. In some embodiments, the piezoelectric actuators disclosed herein are configured to have an operating voltage in a range of 0.1 V to 100 V (e.g., a range of 0.1 V to 30 V) based on a type of actuator and/or a type of sample. In some embodiments, the piezoelectric actuators disclosed herein are configured to have a deflection in the range of 5 nm to 50 μm (e.g., 100 nm-10 μm) based on a type of actuator and/or a type of sample.

(A3) In some embodiments of A1 or A2, one or more actuators of the first set of piezoelectric actuators and/or the second set of piezoelectric actuators have a rounded shape. In some embodiments, at least some of the piezoelectric actuators described herein have a circular shape or an oval shape. In some embodiments, at least some of the piezoelectric actuators described herein have a non-rounded shape (e.g., square or rectangular).

(A4) In some embodiments of any of A1-A3, one or more actuators of the third set of piezoelectric actuators have an annular shape configured to eject via a center of the annular shape. In some embodiments, the third set of piezoelectric actuators are arranged on a membrane over the outlet channel. In some embodiments, the third set of piezoelectric actuators have a rounded shape. In some embodiments, the third set of piezoelectric actuators are MEMS actuators. In some embodiments, the third set of piezoelectric actuators are configured for ejecting droplets containing particles into another system for further analysis.

(A5) In some embodiments of any of A1-A4, at least one of the first set of piezoelectric actuators, the second set of piezoelectric actuators, and the third set of piezoelectric actuators is configured to operate in a frequency range between 1 kHz and 100 GHz. In some embodiments, operating in the frequency range comprises vibrating in the frequency range. In some embodiments, at least some of the piezoelectric actuators described herein are configured to adjust the operating frequency within the frequency range based on the type of particles in the fluidic sample and/or the intended operation (e.g., dissociation, levitation, rotation, and/or mixing). As an example, a MHz subrange may be used to levitate particles (e.g., cells), a kHz and/or MHz subrange may be used to rotate particles, and a kHz range may be used to sort particles. As another example, the first set of piezoelectric actuators may operate in a kHz-MHz range to perform ultrasonication of a sample, where the selected frequency within the range may depend on the desired function (e.g., disrupt clumps, disrupt clogs and/or push/stimulate fluid flow) and the types of particles in the sample. In some embodiments, a frequency at a low end of the range (e.g., 20 kHz) is used for a first ultrasonication operation, and if the first ultrasonication operation is not successful (e.g., clumping is still present in the sample) then the frequency is increased (e.g., to 200 kHz) and a second ultrasonication operation is performed. In some embodiments, the ultrasonication operation is repeated at increasing frequencies until the operation is determined to be successful.

(A6) In some embodiments of any of A1-A5, at least one of the first set of piezoelectric actuators, the second set of piezoelectric actuators, and the third set of piezoelectric actuators is arranged on a membrane. For example, the membrane may be composed of a thin layer of substrate (e.g., thickness in the range of 1 μm to 5 μm).

(A7) In some embodiments of any of A1-A6, the first set of piezoelectric actuators comprise one or more actuators having a first size, and the second set of piezoelectric actuators comprise one or more actuators having a second size, different than the first size. For example, the actuators of the first set of actuators may have a size that is two or three times the size of the actuators of the second set of actuators.

(A8) In some embodiments of any of A1-A7, the microfluidic device further includes an optical layer (e.g., the optical layer 152), wherein the inlet channel is defined by the optical layer. In some embodiments, the optical layer is composed of a transparent or translucent material such as glass. For example, the optical layer may be composed of glass. In some embodiments, the optical layer comprises a top layer. In some embodiments, the optical layer is composed of a transparent and/or translucent material.

(A9) In some embodiments of any of A1-A8, the microfluidic device further includes a passivation layer (e.g., the passivation layer 156) separating the first, second, and third sets of piezoelectric actuators from the fluidic sample in the microfluidic channel. In some embodiments, the passivation layer is composed of nitride, silicon nitride, silicon carbide, SiO2, aluminum nitride, aluminum oxide, and/or a photo-imagable polymer. For example, the passivation layer may be composed of nitride. In some embodiments, the passivation layer has a thickness in the range of 0.01 μm-1 μm. Example passivation materials include silicon nitride, silicon carbide, SiO2, aluminum nitride, aluminum oxide, and photo-imagable polymers.

(A10) In some embodiments of any of A1-A9, the microfluidic device further includes a passivation layer (e.g., the passivation layer 156) arranged between the set of piezoelectric actuators and the microfluidic channel. For example, the passivation layer may be composed of nitride. In some embodiments, the passivation layer has a thickness in the range of 0.01 μm-1 μm. Example passivation materials include silicon nitride, silicon carbide, SiO2, aluminum nitride, aluminum oxide, and photo-imageable polymers.

(A11) In some embodiments of any of A1-A10, the microfluidic device further includes an intermediate adhesion layer arranged between the set of piezoelectric actuators and the passivation layer. In some embodiments, the intermediate adhesion layer is configured to match a mechanical impedance between the piezoelectric layer 178 and the passivation layer 156. In some embodiments, the intermediate adhesion layer is configured to reduce cracking in the passivation layer 156. In some embodiments, the intermediate adhesion layer is configured to impedance match between the set of piezoelectric actuators and the passivation layer. In some embodiments, the intermediate adhesion layer is composed of metal, such as Titanium (Ti). In some embodiments, the intermediate adhesion layer is configured to reduce cracking in the passivation layer.

(A12) In some embodiments of A1-A11, the intermediate adhesion layer is composed of titanium. For example, the titanium has a thickness in the range of 10 nm-300 nm.

(A13) In some embodiments of A1-A12, the microfluidic device further includes a polymer layer (e.g., the polymer layer 154) that defines at least a portion of the microfluidic channel. In some embodiments, the polymer layer is configured to bond the substrate to the optical layer.

(A14) In some embodiments of A1-A13, the microfluidic device further includes one or more electrodes arranged adjacent to the microfluidic channel between the inlet and the outlet and configured to apply an electrical field to the fluidic sample in the microfluidic channel and/or sense one or more properties of the fluidic sample. In some embodiments, the first and second sets of piezoelectric actuators and the one or more electrodes are arranged on a same layer. In some embodiments, the one or more electrodes have a thickness in the range of 0.01 μm-1 μm.

(A15) In some embodiments of A1-A14, the microfluidic device further includes control circuitry (e.g., the control circuitry described in reference to FIG. 3) electrically coupled to the first and second sets of piezoelectric actuators and configured to provide actuation signals to the first, second, and third sets of piezoelectric actuators (e.g., piezoelectric actuators 301). In some embodiments, the control circuitry is further configured to provide activation signals to one or more electrodes (e.g., electrodes 305) to selectively charge particles of the fluidic sample flowing through the microfluidic channel and/or selectively sense at least one property of the fluidic sample. In some embodiments, the control circuitry comprises one or more microcontrollers (e.g., processor(s) 302). In some embodiments, the control circuitry monitors signals from one or more sensing elements (e.g., piezoelectric actuator(s) 354 in a sensing mode and/or electrode(s) 352) and adjusts operation of the actuators accordingly. For example, if the sensing signals indicate that a clump is present the actuators are configured to break up the clump.

(A16) In some embodiments of A1-A15, the microfluidic device further comprises a set of electrodes (e.g., the electrodes 352), wherein the set of electrodes is configured to provide actuation signals, from the control circuitry, to at least one of the first set of piezoelectric actuators and the second set of piezoelectric actuators. In some embodiments, the operation of the set of electrodes is based on the operation of the actuators (e.g., the electrode operation is timed to reduce/minimize interference from operation of the actuators).

(B1) In another aspect, some embodiments include a method (e.g., the method 400) that includes: (i) providing a fluidic sample comprising plurality of particles (e.g., cells) through an inlet (e.g., inlet 103) to a microfluidic channel (e.g., fluid channel 102) of a microfluidic device (e.g., the microfluidic device 100), the microfluidic channel having an outlet (e.g., one of the plurality of outlets 107a-107c), (ii) selectively dissociating two or more particles of the fluidic sample using a first set of piezoelectric (e.g., the first set of piezoelectric actuators 105) actuators positioned adjacent to the inlet, (iii) selectively manipulating, using a second set of piezoelectric actuators (e.g., the second set of piezoelectric actuators 121a-121b), one or more particles of the fluidic sample flowing through the microfluidic channel, and (iv) selectively ejecting, with a third set of piezoelectric actuators (e.g., the third set of piezoelectric actuators 109a-109c) located adjacent to the outlet, a portion of the fluidic sample from the microfluidic channel. In some embodiments, the third set of piezoelectric actuators and the outlet are sized to be able to eject particles with a diameter ranging from 2 μm-50 μm. In some embodiments, the method further comprises inducing, using the first and/or second set of piezoelectric actuators, a laminar flow from the inlet of the microfluidic channel toward the outlet. In some embodiments, at least one of the first and second sets of piezoelectric actuators are fabricated using a combination of SOI PZT with dry reactive ion etch (DRIE), polymer laminate microfluidics and top-glass adhesion to the polymer.

(B2) In some embodiments of B1, the method further includes selectively providing actuation signals (e.g., via the actuation circuitry 330) to the first, second, and third sets of piezoelectric actuators based on obtained sensing data (e.g., via the measurement/analysis circuitry 350). In some embodiments, the actuation signals are generated to create an oscillation frequency in the actuators that is based on a fluid composition (e.g., size and/or concentration of particles) in the microfluidic channel. For example, the third set of actuators may be configured to oscillate such that only 1 cell is ejected at a time. For example, the sense signals may include information about the state of the fluid.

(B3) In some embodiments of B1-B2, the method further includes determining a state of the microfluidic device, including: (i) operating one or more actuators of the first set of piezoelectric actuators and/or the second set of piezoelectric actuators in an actuation state to produce a vibration signal, and (ii) switching operation of the one or more actuators to a sensing state to sense an echo response corresponding to the vibration signal. For example, if there is a large clump of cells sitting on the one or more actuators, then its response will change. In some embodiments, a first subset of actuators operate in the actuation state to produce one or more vibration signals and a second subset of actuators operate in the sensing state to sense echo responses of the one or more vibration signals. As an example, the acoustic signal received by the piezoelectric actuators after a rebound indicates a viscosity and/or density of the sample and can be used to assess the agglomeration/coagulation/clogging of the sample.

(B4) In some embodiments of B1-B3, selectively manipulating, using the second set of piezoelectric actuators, the one or more particles of the fluidic sample flowing through the microfluidic channel comprises selectively levitating, rotating, and/or sorting the one or more particles.

(B5) In some embodiments of B1-B4, selectively manipulating, using the second set of piezoelectric actuators, the one or more particles of the fluidic sample flowing through the microfluidic channel comprises adjusting a frequency of operation of the second set of piezoelectric actuators to perform different types of manipulation.

(B6) In some embodiments of B1-B5, the method further includes (i) sensing, via a sense component, a flow rate for the fluidic sample and one or more properties of the one or more particles of the fluidic sample flowing through the microfluidic channel; (ii) determining operating parameters for the first set of piezoelectric actuators and the third set of piezoelectric actuators according to at least the sensed flow rate; and (iii) determining operating parameters for the second set of piezoelectric actuators according to at least the sensed one or more properties of the one or more particles. In some embodiments, the sense component comprises a set of electrodes (e.g., electrodes 352) and/or the second set of piezoelectric actuators (e.g., piezoelectric actuators 354). In some embodiments, the operating parameters include a frequency of operation (e.g., an oscillation frequency). In some embodiments, the one or more properties of the one or more particles includes a measured electrical impedance of each particle and/or a size of each particle.

It will be understood that, although the terms first, second, etc. may be used herein to describe various elements, these elements should not be limited by these terms. These terms are only used to distinguish one element from another. For example, a first array could be termed a second array, and, similarly, a second array could be termed a first array, without departing from the scope of the various described embodiments. The first array and the second array are both arrays, but they are not the same array.

The terminology used in the description of the embodiments herein is for the purpose of describing particular embodiments only and is not intended to be limiting of the scope of claims. As used in the description and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The foregoing description, for purpose of explanation, has been described with reference to specific embodiments. However, the illustrative discussions above are not intended to be exhaustive or to limit the scope of claims to the precise forms disclosed. Many modifications and variations are possible in view of the above teachings. The embodiments were chosen and described in order to best explain the principles of the various described embodiments and their practical applications, to thereby enable others skilled in the art to best utilize the principles and the various described embodiments with various modifications as are suited to the particular use contemplated.

What is claimed is:

1. A microfluidic device, comprising:
an inlet channel;
an outlet channel;
a microfluidic channel arranged on a substrate between the inlet channel and the outlet channel such that an outlet of the microfluidic channel is positioned above at least a portion of the outlet channel;
a first set of piezoelectric actuators arranged adjacent to the inlet channel and configured to dissociate particles of a fluidic sample in the microfluidic channel;
a second set of piezoelectric actuators arranged between the inlet channel and the outlet channel, the second set of piezoelectric actuators configured to manipulate the particles of the fluidic sample as the particles move through the microfluidic channel; and
a third set of piezoelectric actuators arranged above the outlet channel and adjacent to the outlet, the third set of piezoelectric actuators configured to eject a portion of the fluidic sample out of the microfluidic channel via the outlet.

2. The microfluidic device of claim 1, wherein at least one of the first set of piezoelectric actuators, the second set of piezoelectric actuators, and the third set of piezoelectric actuators comprises one or more micro-electro-mechanical system (MEMS) actuators.

3. The microfluidic device of claim 1, wherein one or more actuators of the first set of piezoelectric actuators and/or the second set of piezoelectric actuators have a rounded shape.

4. The microfluidic device of claim 1, wherein one or more actuators of the third set of piezoelectric actuators have an annular shape configured to eject via a center of the annular shape.

5. The microfluidic device of claim 1, wherein at least one of the first set of piezoelectric actuators, the second set of piezoelectric actuators, and the third set of piezoelectric actuators is configured to operate in a frequency range between 1 kilohertz and 100 gigahertz.

6. The microfluidic device of claim 1, wherein at least one of the first set of piezoelectric actuators, the second set of piezoelectric actuators, and the third set of piezoelectric actuators is arranged on a membrane.

7. The microfluidic device of claim 1, wherein the first set of piezoelectric actuators comprise one or more actuators having a first size, and the second set of piezoelectric actuators comprise one or more actuators having a second size, different than the first size.

8. The microfluidic device of claim 1, further comprising an optical layer, wherein the inlet channel is defined by the optical layer.

9. The microfluidic device of claim 1, further comprising a passivation layer separating the first, second, and third sets of piezoelectric actuators from the fluidic sample in the microfluidic channel.

10. The microfluidic device of claim 1, further comprising a passivation layer arranged between the set of piezoelectric actuators and the microfluidic channel.

11. The microfluidic device of claim 10, further comprising an intermediate adhesion layer arranged between the set of piezoelectric actuators and the passivation layer.

12. The microfluidic device of claim 11, wherein the intermediate adhesion layer is a metal layer.

13. The microfluidic device of claim 1, further comprising a polymer layer that defines at least a portion of the microfluidic channel.

14. The microfluidic device of claim 1, further comprising one or more electrodes arranged adjacent to the microfluidic channel between the inlet channel and the outlet channel and configured to apply an electrical field to the fluidic sample in the microfluidic channel and/or sense one or more properties of the fluidic sample.

15. The microfluidic device of claim 1, further comprising control circuitry electrically coupled to the first and second sets of piezoelectric actuators and configured to provide actuation signals to the first, second, and third sets of piezoelectric actuators.

16. A method performed at a microfluidic device, the method comprising:
providing a fluidic sample comprising a plurality of particles through an inlet to a microfluidic channel of the microfluidic device, the microfluidic channel having an outlet;
selectively dissociating two or more particles of the fluidic sample using a first set of piezoelectric actuators positioned adjacent to the inlet;
selectively manipulating, using a second set of piezoelectric actuators, one or more particles of the fluidic sample flowing through the microfluidic channel; and
selectively ejecting, with a third set of piezoelectric actuators located adjacent to the outlet, a portion of the fluidic sample from the microfluidic channel.

17. The method of claim 16, further comprising determining a state of the microfluidic device, including:
operating one or more actuators of the first set of piezoelectric actuators and/or the second set of piezoelectric actuators in an actuation state to produce a vibration signal; and
switching operation of the one or more actuators to a sensing state to sense an echo response corresponding to the vibration signal.

18. The method of claim 16, wherein selectively manipulating, using the second set of piezoelectric actuators, the one or more particles of the fluidic sample flowing through the microfluidic channel comprises adjusting a frequency of operation of the second set of piezoelectric actuators to perform different types of manipulation.

19. The method of claim 16, further comprising selectively providing, via control circuitry, actuation signals to the first, second, and third sets of piezoelectric actuators based on obtained sensing data.

20. The method of claim 19, further comprising:
sensing, via a sense component, a flow rate for the fluidic sample and one or more properties of the one or more particles of the fluidic sample flowing through the microfluidic channel;
determining a first set of operating parameters for operating the first set of piezoelectric actuators and the third set of piezoelectric actuators according to at least the sensed flow rate; and
determining a second set of operating parameters for operating the second set of piezoelectric actuators according to at least the sensed one or more properties of the one or more particles; and
wherein the actuation signals are selectively provided in accordance with the determined first and second sets of operating parameters.
